(19) United States
(12) Patent Application Publication
(10) Pub. No.: US 2024/0301614 A1
(43) Pub. Date: Sep. 12, 2024

(54) STEAM SPRAY DEVICE AND CLOTHING MANAGEMENT DEVICE INCLUDING SAME

(71) Applicant: SAMSUNG ELECTRONICS CO., LTD., Suwon-si Gyeonggi-do (KR)

(72) Inventors: Woojin KIM, Suwon-si (KR); Eungryeol SEO, Suwon-si (KR)

(73) Assignee: SAMSUNG ELECTRONICS CO., LTD., Suwon-si Gyeonggi-do (KR)

(21) Appl. No.: 18/666,240

(22) Filed: May 16, 2024

Related U.S. Application Data
(63) Continuation of application No. PCT/KR2022/018067, filed on Nov. 16, 2022.

(30) Foreign Application Priority Data
Jan. 6, 2022 (KR) 10-2022-0002219

(51) Int. Cl. D06F 58/20; D06F 58/10; D06F 87/00

(52) U.S. Cl. CPC D06F 58/203; D06F 58/10; D06F 87/00

(57) ABSTRACT

A clothing management device according to an embodiment may include: an outer case forming the exterior thereof; an inner case arrangeable inside the outer case to a clothing accommodation space; a steam generation device for heating water to generate steam; a steam nozzle which is coupled to the inner case and sprays steam; and a steam hose for connecting the steam generation device and the steam nozzle, wherein the steam nozzle includes: a steam spray hole for spraying steam into the clothing accommodation space; a main fluid channel directly connectable between the steam hose to the steam spray hole; and a side fluid channel branched from the main fluid channel so that at least a part of the steam turns a direction in which the steam moves in the steam nozzle.

### CROSS-REFERENCE TO RELATED APPLICATIONS

This application is a continuation of International Application PCT/KR2022/018067, filed Nov. 16, 2022, and claims foreign priority to Korean Application 10-2022-0002219, filed Jan. 6, 2022, the disclosures of which are incorporated herein by reference in their entireties.

### TECHNICAL FIELD

Various embodiments described herein relate to a steam spray device and a clothing management device including the steam spray device.

### BACKGROUND ART

A clothing management device, which is a device that cares for or manages clothes by housing them in a clothing accommodation space, may remove wrinkles, dust, or odors from the clothes or dry or sanitizes the clothes. More recently, the clothing management device has been provided for home use and is used to manage clothes such as shirts, pants, and coats. For example, the clothing management device may include a fan and a fluid channel structure to generate an air flow toward clothes mounted in the clothing accommodation space or may include a steam spray device to discharge steam onto the clothes.

The steam spray device included in the clothing management device may spray steam onto clothes to remove wrinkles from the clothes or remove residual contaminants from the clothes. For example, upon contact between steam and a contaminant, the contaminant may be dissolved by the steam in a hot liquid state and removed as being collected at an outlet provided at the bottom of the clothing accommodation space along with the steam.

### SUMMARY

According to an embodiment of the present disclosure, there is provided a clothing management device including: an outer case forming an exterior of the clothing management device; an inner case arrangeable inside the outer case such that while the inner case is arranged inside the outer case, a clothing accommodation space is provided; a steam generation device to generate steam by heating water; a steam nozzle to be coupled to the inner case to spray steam; and a steam hose to connect the steam generation device and the steam nozzle, wherein the steam nozzle includes: a steam spray hole to spray steam generated by the steam generation device into the clothing accommodation space; a main fluid channel directly connectable between the steam hose and the steam spray hole; and a side fluid channel branched from the main fluid channel to allow at least a portion of the steam to turn a direction in which the steam is moved inside the steam nozzle.

According to another embodiment of the present disclosure, there is provided a steam spray device including: a steam generation device to generate steam by heating water; a steam hose to connect to the steam generation device; and a steam nozzle to be connected to the steam hose to receive steam generated by the steam generation device and spray the steam to an outside of the steam nozzle, wherein the steam nozzle includes: a steam spray hole adapted to spray steam to the outside of the steam nozzle; a main fluid channel directly connectable between the steam hose and the steam spray hole; a side fluid channel branched from the main fluid channel to allow at least a portion of steam to turn inside the steam nozzle; and a partitioning including a first side wall facing the main fluid channel and a second side wall facing the side fluid channel, and the partitioning being between the main fluid channel and the side fluid channel.

According to various embodiments, a steam spray device and a clothing management device including the steam spray device may include a main fluid channel and a side fluid channel to guide a movement direction of steam in which the steam moves, and may provide a high spray pressure of a steam nozzle to allow steam sprayed from the stem nozzle to be sprayed in a wide direction and sprayed evenly onto clothes.

### BRIEF DESCRIPTION OF DRAWINGS

FIG. 1 is a perspective view of a clothing management device according to an embodiment of the present disclosure.

FIG. 2 is a block diagram illustrating a configuration of a clothing management device according to an embodiment of the present disclosure.

FIG. 3 is a side view of a clothing management device according to an embodiment of the present disclosure.

FIG. 4 is a perspective view of a steam nozzle according to an embodiment of the present disclosure.

FIG. 5 is a top view of a steam nozzle according to an embodiment of the present disclosure.

FIG. 6 is a graph illustrating a change in an X-direction spray speed of a steam nozzle according to an embodiment of the present disclosure.

FIG. 7A is a diagram illustrating a state in which a steam nozzle sprays steam according to an embodiment of the present disclosure.

FIG. 7B is a diagram illustrating a state in which a steam nozzle sprays steam according to an embodiment of the present disclosure.

FIG. 7C is a diagram illustrating a state in which a steam nozzle sprays steam according to an embodiment of the present disclosure.

FIG. 7D is a diagram illustrating a state in which a steam nozzle sprays steam according to an embodiment of the present disclosure.

### DETAILED DESCRIPTION

Hereinafter, embodiments will be described in detail with reference to the accompanying drawings. When describing the embodiments with reference to the accompanying drawings, like reference numerals refer to like components and a repeated description related thereto will be omitted.

It should be appreciated that various embodiments of the present disclosure and the terms used therein are not intended to limit the technological features set forth herein to some particular embodiments but include various changes, equivalents, or replacements for a corresponding embodiment. In connection with the description of the drawings, like reference numerals may be used for similar or related components. It is to be understood that a singular form of a noun corresponding to an item may include one or more of the things unless the relevant context clearly indicates otherwise. As used herein, “A or B,” “at least one of A and B,” “at least one of A or B,” “A, B or C,” “at least one of A, B and C,” and “A, B, or C,” each of which may include any one of the items listed together in the corresponding one of the phrases, or all possible combinations thereof. Terms such as “first” or “second,” or “1st” or “2nd” may simply be used to distinguish the component from other components in question, and do not limit the components in other aspects (e.g., importance or order). It is to be understood that if an element (e.g., a first element) is referred to, with or without the term “operatively” or “communicatively,” as “coupled with,” “coupled to,” “connected with,” or “connected to” another element (e.g., a second element), it means that the element may be coupled with the other element directly (e.g., by wire), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry.” A module may be a single integral component, or a minimum unit or part thereof, adapted to perform one or more functions. For example, according to an embodiment, the module may be implemented in the form of an application-specific integrated circuit (ASIC).

Various embodiments set forth herein may be implemented as software (e.g., a program) including one or more instructions that are stored in a storage medium (e.g., an internal memory or an external memory) that is readable by a machine (e.g., an electronic device). For example, a processor of the machine (e.g., the electronic device) may invoke at least one of the one or more instructions stored in the storage medium and execute it. This allows the machine to be operated to perform at least one function according to the at least one instruction invoked. The one or more instructions may include code generated by a complier or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to various embodiments, a method according to an embodiment of the disclosure may be included and provided in a computer program product. The computer program product may be traded as a product between a seller and a buyer. The computer program product may be distributed in the form of a machine-readable storage medium (e.g., a compact disc read-only memory (CD-ROM)), or be distributed (e.g., downloaded or uploaded) online via an application store (e.g., PlayStore™) or between two user devices (e.g., smartphones) directly. If distributed online, at least part of the computer program product may be temporarily generated or at least temporarily stored in the machine-readable storage medium, such as a memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components or operations may be omitted, or one or more other components or operations may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

A steam nozzle of a steam spray device may be installed at a fixed position in a clothing accommodation space to spray steam onto clothes. However, the steam may not be evenly sprayed or distributed in some areas of the clothes due to various factors, such as, for example, the number of pieces of clothing in the clothing accommodation space, the size of clothes, the type of clothes, or the position of clothes in the clothing accommodation space and, accordingly, a wrinkle removal effect or sterilization and deodorization effects of the steam may be reduced.

A steam spray device and a clothing management device including the steam spray device may require a structural design of a steam nozzle that allows the steam nozzle to spray steam in wider directions from a fixed position and provide a high spray pressure. Various embodiments of the present disclosure provide a steam nozzle adapted to spray steam in a wide direction while the steam is moving through main fluid and side fluid channels and provide a high spray pressure.

The technical goals to be achieved through the various embodiments disclosed herein are not limited to the technical goals described above, and other technical goals not described above may also be clearly understood by a person having ordinary skill in the art to which the present disclosure pertains from the following description.

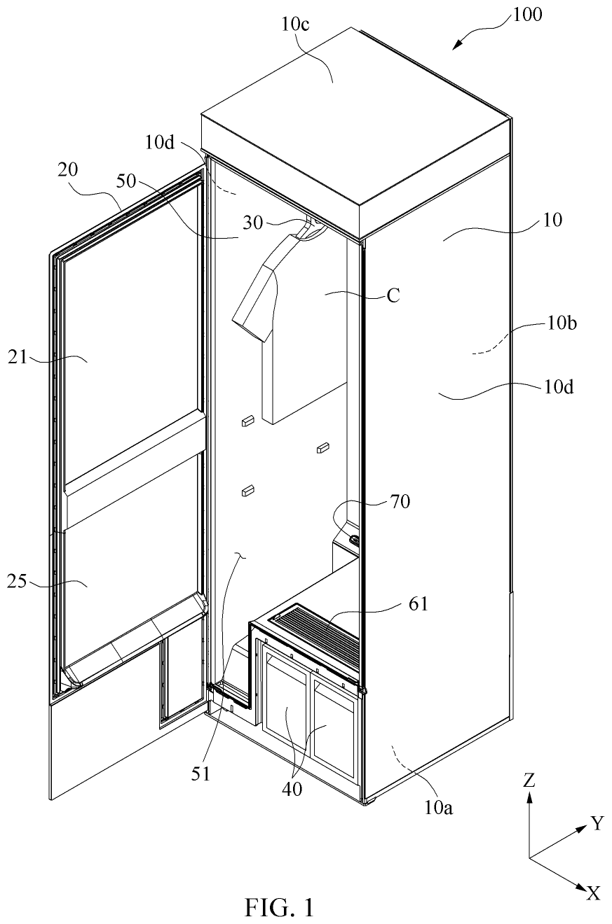

Hereinafter, various embodiments of the present disclosure will be described in detail with reference to FIGS. 1 to 7D.

FIG. 1 is a perspective view of a clothing management device 100 according to an embodiment of the present disclosure.

Referring to FIG. 1, the clothing management device 100 according to an embodiment may include an outer case 10 and a door 20.

In an embodiment, the clothing management device 100 may be a device for caring for or managing, or cleaning clothes C. The clothing management device 100 may include an inner case 50 for receiving therein the clothes C and the door 20 rotatably mounted to open and close the inner case 50, and may receive the clothes C in a clothing accommodation space 51. The clothing management device 100 may spray at least some of wind, hot air, steam, or fragrance onto the clothes C received in the inner case 50 to remove wrinkles, dust, or odors from the clothes C, or dry and sanitize the clothes C.

Although one embodiment of the clothing management device 100 is described herein as a clothing manager installed for home use as an example, actual implementations are not limited thereto, and the clothing management device 100 may be implemented as various types of clothing management devices such as a drying machine or a washing machine. Hereinafter, for ease of description, a target to be processed by the clothing management device 100 will be referred to as “clothes C,” but actual implementations are not limited thereto, and there may be various targets such as towels, blankets, or curtains.

In an embodiment, the outer case 10 may form the exterior of the clothing management device 100 and, inside thereof, the inner case 50 providing the clothing accommodation space 51 receiving therein the clothes C and other components for handling the clothes C may be arranged. In an embodiment, the outer case 10 may include a first surface 10a in a direction (e.g., −Z direction) in which the outer case 10 faces an external support surface (e.g., ground or indoor mounted surface); a second surface 10b facing a direction (e.g., +Y direction) in which the outer case 10 faces the door 20 with the door 20 closed; a third surface 10c facing a ceiling direction (e.g., +Z direction) opposite the first surface 10a; and a plurality of side surfaces 10d in both directions (e.g., +/−X direction) extending from the first surface 10a to the third surface 10c.

In an embodiment, the door 20 may open and close the inner case 50, and when the door 20 is open, the clothes C may be placed into and removed from the inner case 50 inside the outer case 10. The door 20 may include a first door member 21 and a water reservoir member 25 that are coupled to a side facing the inner case 50 in a closed state. For example, the first door member 21 may include glass, a mirror, or a panel, and the second door member 25 may be a frame of the door 20, in which a door guide may be formed to guide condensate formed in the door 20. However, the structure and functions of the door 20 are not limited to the foregoing but may be implemented in various ways.

In an embodiment, the inner case 50 may include at least one support member 30 provided to hold the clothes C thereon. The support member 30 may be provided in a shape of a hanger to support the clothes C. In an embodiment, the interior of the support member 30 may be open to allow air to flow into the interior, and the air may flow into the interior of the clothes C mounted on the support member 30 to remove dust or dirt.

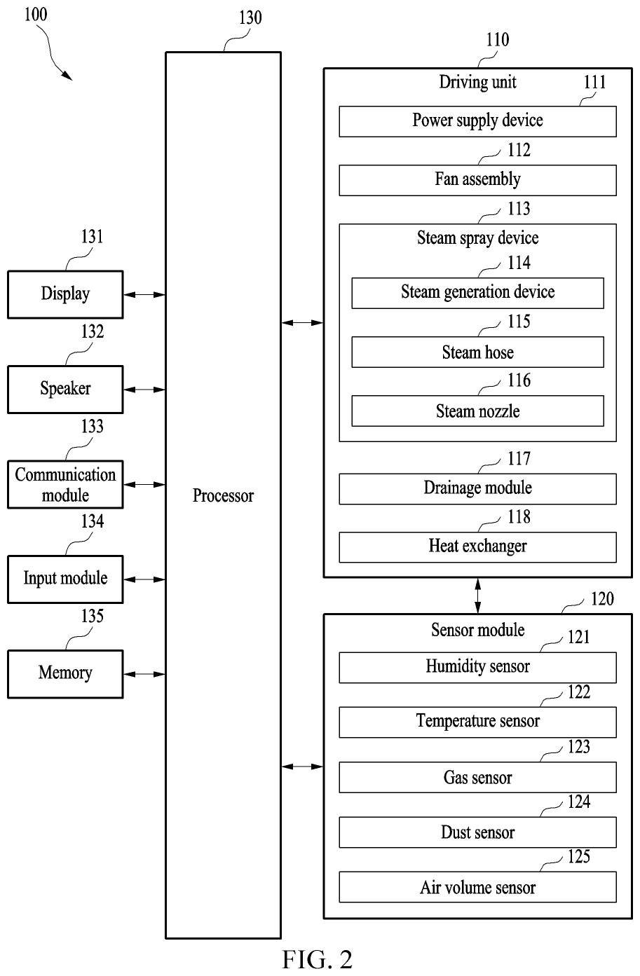

In an embodiment, a water reservoir 40 that is removably arranged from the outer case 10 may be provided in a direction of the first surface 10a of the outer case 10. The water reservoir 40 may be a water supply reservoir for supplying water to a steam generation device (e.g., a steam generation device 114 in FIG. 2) to generate steam or a drain reservoir for draining condensate generated in a heat exchanger (e.g., a heat exchanger 118 in FIG. 2) to the outside.

In an embodiment, a machine chamber may be provided behind the water reservoir 40, and the machine chamber may house various mechanical components necessary to drive the clothing management device 100.

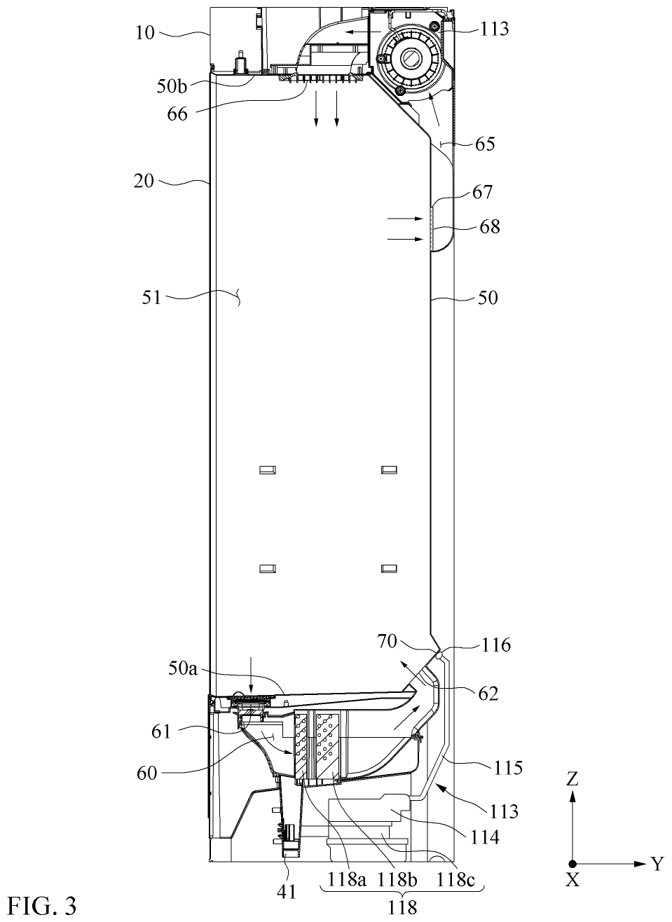

In an embodiment, inside the inner case 50, a first intake vent 61 may be provided to draw in air from the clothing accommodation space 51, and a steam spray hole 70 of a steam nozzle 116 may be coupled to spray steam into the inner case 50. The first intake vent 61 and the steam spray hole 70 will be described in more detail below with reference to FIG. 3 and subsequent drawings.

FIG. 2 is a block diagram illustrating a configuration of the clothing management device 100 according to an embodiment of the present disclosure.

Referring to FIG. 2, the clothing management device 100 may include at least some of a driving unit 110, a sensor module 120, and a processor 130.

In an embodiment, the clothing management device 100 may be implemented with a combination of one or more configurations or features of the previously described embodiments unless it is technically obviously impossible.

In an embodiment, the driving unit 110 may be a component provided to perform the overall mechanical operation of the clothing management device 100 and may perform various operations under the control of the processor 130.

In an embodiment, a power supply device 111 may provide power to the driving unit 110 and other components of the clothing management device 100. The power supply device 111 may provide power to a fan assembly 112 and may control the driving of the fan assembly 112. For example, the power supply device 111 may control the speed of the fan assembly 112 using a voltage control method or a frequency conversion method according to a control signal from the processor 130.

In an embodiment, the power supply device 111 may be controlled by an intelligent power module (IPM) configured as a switching device. The IPM may be a power module to which a protection function for protecting a power device or a drive circuit is added. For example, the power supply device 111 adapted to control a heat exchanger 118 may prevent components of the power supply device 111 and the clothing management device 100 from being damaged by overheating from an increased load.

In an embodiment, the fan assembly 112 may be installed in a circulation channel (e.g., a second circulation channel 65 in FIG. 3) and may induce an air flow in the clothing accommodation space 51. When the fan assembly 112 is driven, air in the clothing accommodation space 51 may circulate along the circulation channel 65, and dust may be removed, and/or the air may be heated and/or dried.

In an embodiment, a steam spray device 113 may generate steam and spray the steam. The steam spray device 113 may include a steam generation device 114 adapted to generate steam, a steam hose 115 connected to the steam generation device 114 to receive steam, and a steam nozzle 116 connected to the steam hose 115 to receive steam and spray the steam.

In an embodiment, the steam spray device 113 may spray steam onto the clothes C to remove contaminants adhering to the clothes C. For example, when steam contacts a contaminant, the contaminant may be dissolved by the steam in a hot liquid state, or the steam may condense and be collected at an outlet provided on a bottom surface of the inner case 50.

In an embodiment, the steam generation device 114 may receive water via the water reservoir 40 and heat the water to generate steam. The steam generation device 114 may transfer steam to the steam nozzle 116 through the steam hose 115, and the steam nozzle 116 may spray or inject hot steam onto the clothes C mounted in the inner case 50 through a steam spray hole (e.g., the steam spray hole 70 in FIG. 1) that is open toward the clothing accommodation space 51 of the inner case 50.

In an embodiment, the steam nozzle 116 may also spray, into the inner case 50, various liquids, such as, for example, cleansing solution, fragrance, and the like, in addition to steam. For example, the steam nozzle 116 may be connected to a cleansing fluid supply device (not shown) and/or a fragrance supply device (not shown) to spray a cleansing fluid to clean any residual contaminants on the clothes C or spray fragrance to deodorize or make the clothes C fragrant.

In an embodiment, a drainage module 117 may drain water generated in the clothing management device 100 to the outside. For example, the drainage module 117 may be connected to a water reservoir (e.g., the water reservoir 40 in FIG. 1) to drain condensate generated in the heat exchanger 118 and/or dirty water collected in the clothing accommodation space 51.

In an embodiment, the heat exchanger 118 may remove moisture from, or heat, air transferred from the inner case 50. For example, the heat exchanger 118 may dry or heat the air passing through the heat exchanger 118 as a refrigerant condenses or expands, and may provide the dried hot air to the inner case 50. The configuration and operations of the heat exchanger 118 will be described in more detail below with reference to FIG. 3.

In an embodiment, the sensor module 120 may sense an operating state or a surrounding environment of the clothing management device 100, and generate and output electrical signals of sensing results. The sensor module 120 may transmit the electrical signals to the processor 130 and/or store the sensing results in a memory 135 of the clothing management device 100 or in an external device. For example, the sensor module 120 may generate electrical signals or obtain data by sensing an operating state or a surrounding environment of the clothing management device 100 during a dry course, and the processor 130 may process the signals or data received from the sensor module 120 to obtain diagnostic information.

In an embodiment, sensors included in the sensor module 120 may be implemented as physically separate devices or each of the sensors may be implemented as a single device. The sensor module 120 is not limited to being implemented as a single physical device. The sensor module 120 may transmit sensing values to the processor 130, and the processor 130 may control the operations of the clothing management device 100 based on the received sensing values, store them in the memory 135 as diagnostic information, or transmit them to an external device (e.g., a server and a smartphone) via a communication module 133 to store them in the external device.

In an embodiment, the sensor module 120 may include at least some of a humidity sensor 121, a temperature sensor 122, a gas sensor 123, a dust sensor 124, and an air volume sensor 125.

In an embodiment, the humidity sensor 121 and the temperature sensor 122 may sense the humidity and temperature of the clothing accommodation space 51 of the clothing management device 100, respectively, or the humidity and temperature of an environment around the clothing management device 100, respectively.

In an embodiment, the humidity sensor 121 may include a hygroscopic member, such as a porous ceramic or polymeric membrane, and in this case, the hygroscopic member may absorb ambient air to detect a change in resistance or capacitance or to detect a relative humidity of ambient air based on a change in resonant frequency by a change in weight.

In an embodiment, the temperature sensor 122 may be implemented as a thermistor which is a type of resistor using a property that the resistance of a material changes with temperature, and in this case, the thermistor may have a negative temperature coefficient (NTC) characteristic by which the resistance decreases as the temperature increases and increases as the temperature decreases.

In an embodiment, the temperature sensor 122 may further include a temperature adjustment device such as a thermostat, and in this case, the thermostat may detect an amount of heat generated in the heat exchanger 118 and control the temperature of air supplied to the inner case 50 to be maintained at a specific temperature.

In an embodiment, the gas sensor 123 and the dust sensor 124 may measure a contamination level of air. For example, the gas sensor 123 may be a volatile organic compounds (VOCs) sensor and may measure the concentration of gases such as hydrogen, hydrogen sulfide, ammonia, ethanol, carbon monoxide, methane, and propane in the air of the clothing accommodation space 51. For example, the dust sensor 124 may measure the concentration of fine dust and ultra-fine dust in the air of the clothing accommodation space 51.

In an embodiment, the air volume sensor 126 may measure an air volume of the fan assembly 112, and the processor 130 may receive the measured air volume to control a driving force of the fan assembly 112 to increase or decrease. In an embodiment, the air volume sensor 126 may measure an air volume in a circulation channel (e.g., a first circulation channel 60 or a second circulation channel 65 in FIG. 3) or an air volume inside at least a portion of the inner case 50.

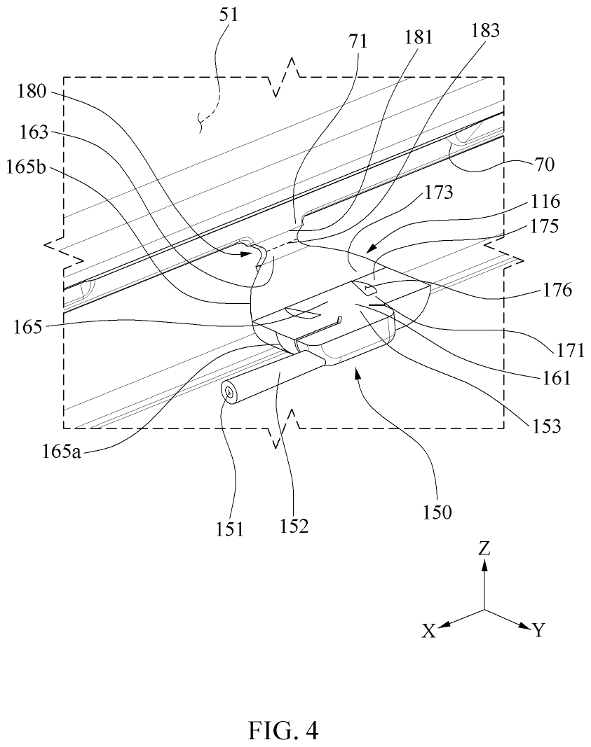

In an embodiment, the sensor module 120 is not limited to the configuration described above, but may further include at least one of a filter sensor (not shown) adapted to sense a state of a filter (e.g., a filter 68 in FIG. 4) detecting a type or remaining amount of detergent, a door sensor (not shown) adapted to sense whether the door 20 is open or closed, and a vibration sensor (not shown) adapted to sense the degree to which the clothing management device 100 vibrates.

In an embodiment, the processor 130 may control the overall operation of the clothing management device 100 and may be a printed circuit board (PCB) on which a plurality of electronic components is mounted. The processor 130 may include, for example, at least some of a random-access memory (RAM), a read-only memory (ROM), a graphics processing unit (GPU), a main central processing unit (CPU), first to n interfaces, and a bus.

In an embodiment, the processor 130 and the memory 135 may be implemented as physically separate components or as a single component in which the processor 130 includes the memory 135. The processor 130 may also be implemented as a single system configured with a single component or a plurality of components. The memory 135 may also be implemented as a single system configured with a single component or a plurality of components.

In an embodiment, when receiving a command, the processor 130 may operate the driving unit 110 and control the driving of the clothing management device 100 such that it performs a plurality of courses in a stepwise or selective manner based on an input signal.

In an embodiment, while the clothing management device 100 is being driven, the sensor module 120 may detect an operating state of the clothing management device 100, and based on this, the processor 130 may provide feedback on the driving of the clothing management device 100 or obtain diagnostic information to display it on a display 131.

In an embodiment, the display 131 may display the diagnostic information obtained through the sensor module 120 under the control of the processor 130. The display 131 may be disposed on the door 20 of the clothing management device 100, allowing a user to check the operating state of the clothing management device 100. Alternatively, the display 131 may be implemented in an external form, rather than built-in form within the clothing management device 100, and may display image data on an external display that is connected, by wire or wirelessly, to the clothing management device 100.

In an embodiment, a speaker 132 may be built into the clothing management device 100 and may output various notifications or voice messages directly as sound, in addition to various audio data obtained through various processing operations such as decoding, amplification, noise filtering, and the like by an audio processing unit (not shown).

In an embodiment, the communication module 133 may communicate with an external device (e.g., a server and a smartphone) to transmit and receive various types of data by various types of communication methods. For example, the communication module 133 may transmit information obtained by the sensor module 120 to a server (or a smartphone), or receive control commands for driving the clothing management device 100 from the server (or the smartphone).

In an embodiment, the communication module 133 may include, for example, at least one of a Bluetooth chip, a Wi-Fi chip, a wireless communication chip, and a near-field communication (NFC) chip that perform wireless communication, and an Ethernet module and a universal serial bus (USB) module that perform wired communication. In this case, the Ethernet module or USB module that performs wired communication may communicate with an external device through input/output ports.

In an embodiment, an input module 134 may be configured to receive various types of user commands from the user and transmit the received user commands to the processor 130. To this end, the input module 134 may include an input device such as a touch panel or key, a plurality of operation buttons, and a rotary lever.

In an embodiment, the memory 135 may store various instructions, programs, or data necessary for the operations of the clothing management device 100 or the processor 130. The memory 135 may store information obtained by the sensor module 120 and data received from an external electronic device.

In an embodiment, the memory 135 may be accessed by the processor 130, and data may be read/written/modified/deleted/updated by the processor 130. Therefore, in describing embodiments of the present disclosure, the term “memory” may include the memory 135, a RAM or a ROM in the processor 130, or a memory card provided in the clothing management device 100.

In an embodiment, the memory 135 may be implemented as a volatile memory such as a static random-access memory (SRAM) and a dynamic random-access memory (DRAM), a non-volatile memory such as a flash memory, a ROM, an erasable programmable read-only memory (EPROM), and an electrically erasable programmable read-only memory (EEPROM), a hard disk drive (HDD), or a solid-state drive (SSD).

FIG. 3 is a side view of the clothing management device 100 according to an embodiment of the present disclosure.

Referring to FIG. 3, the clothing management device 100 according to an embodiment may include a first circulation channel 60, a second circulation channel 65, and a steam spray device 113.

In an embodiment, the clothing management device 100 may be implemented with a combination of one or more configurations or features of the previously described embodiments unless it is technically obviously impossible.

In an embodiment, the inner case 50 may include a bottom surface 50a, and various components of a driving unit (e.g., the driving unit 110 in FIG. 2) for driving the clothing management device 100 may be accommodated between the bottom surface 50a and the outer case 10.

In an embodiment, a channel assembly (e.g., 60 and 65) may communicate to draw air from the inner case 50, process the air, and discharge the processed air back into the inner case 50. The channel assembly (e.g., 60 and 65) may allow air in the inner case 50 to circulate and may continuously apply a strong wind or hot air to clothes C received in the inner case 50. In an embodiment, the channel assembly (e.g., 60 and 65) may include at least some of the first circulation channel 60 and the second circulation channel 65.

In an embodiment, the first circulation channel 60 may include a first intake vent 61 and a first exhaust vent 62 provided on the bottom surface 50a of the inner case 50. In an embodiment, the first intake vent 61 may draw in air from the inner case 50 in a downward direction (e.g., −Z direction). In this case, air entering the first circulation channel 60 may pass through the heat exchanger 118 to be heated and/or dried. In an embodiment, the heated and/or dried air may be discharged into the interior of the inner case 50 through the first exhaust vent 62, and the first circulation channel 60 may heat and/or dry the air in the clothing accommodation space 51.

In an embodiment, at least some components of the heat exchanger 118 may communicate with the first circulation channel 60 and may heat and/or dehumidify the air being drawn into the first intake vent 61. In an embodiment, the heat exchanger 118 may be installed in or communicating with the first circulation channel 60, and may heat and/or dehumidify the transferred air and discharge it to the first exhaust vent 62.

For example, air that has passed through the first circulation channel 60 may be heated and dehumidified to be in a hot dry state, and such hot dry air may be discharged through the first exhaust vent 62 into the interior of the inner case 50. This hot dry air may remove wrinkles from the clothes C or sanitize the clothes C, dry the wet clothes C, or remove water formed by steam sprayed onto the clothes C.

In an embodiment, the first circulation channel 60 may include a connection member 41 that connects the machine chamber and the water reservoir 40. For example, the connection member 41 may receive water from the water reservoir 40, supply water to the steam generation device 114 by being connected to the steam generation device 114, and discharge condensate generated in the heat exchanger 118 to the water reservoir 40 by being connected to the heat exchanger 118.

In an embodiment, the second circulation channel 65 may include a second intake vent 67 provided on one surface facing the door 20 of the inner case 50 and a second exhaust vent 66 provided on a ceiling surface 50b of the inner case 50. In an embodiment, the second intake vent 67 may draw in air from the inner case 50 in a frontal direction (e.g., +Y direction).

In an embodiment, a filter 68 may be provided in the second circulation channel 65 and may collect dust from air drawn into the second intake vent 67. In an embodiment, the filter 68 may be installed in the second intake vent 67 and may filter out dust or fine dust generated in the inner case 50. In an embodiment, the filter 68 may be a filter of various types, and may be adapted to filter out at least some of, for example, dust, fine dust, ultra-fine dust, bacteria, polymeric materials, or viruses.

For example, the second exhaust vent 66 may discharge air in an inward direction of the inner case 50, and the discharged air may dislodge debris or dirt adhering to the clothes C mounted in the clothing accommodation space 51 from the clothes C. Of the debris, relatively lighter dust or fine dust may travel with the air. At least a portion of the air in the inner case 50 may be drawn into the second intake vent 67 and may be filtered out as it passes through the filter 68.

In an embodiment, the heat exchanger 118 may include at least some of an evaporator 118a, a condenser 118b, a compressor 118c, and an expander (not shown). Hereinafter, the operations of dehumidifying and heating air by the components of the heat exchanger 118 will be described in detail.

In an embodiment, the evaporator 118a may evaporate a liquid refrigerant expanded in the expander (not shown). As a result, the evaporator 118a may return a low-temperature and low-pressure individual refrigerant to the compressor 118c. The evaporator 118a may absorb heat from the surroundings through an evaporation process in which a low-pressure liquid refrigerant is changed into a gaseous refrigerant. The evaporator 118a may cool the air passing through the evaporator 118a during the evaporation process. When the ambient air is cooled by the evaporator 118a, and the temperature of the ambient air becomes lower than the dew point, and the air around the evaporator 118a may condense.

In an embodiment, as condensation occurs around the evaporator 118a, the absolute humidity of the air passing through the evaporator 118a may be lowered. For example, the amount of water vapor contained in the air passing through the evaporator 118a may be reduced. Using the condensation occurring around the evaporator 118a, the clothing management device 100 may reduce the amount of water vapor contained in the air of the clothing accommodation space 51, and may also dry and sanitize the clothes C.

In an embodiment, the condenser 118b may condense a compressed gaseous refrigerant into a liquid. The condenser 118b may release heat to the surroundings through such a condensation process of the refrigerant. The condenser 118b may heat air through the heat generated in the condensation process of the refrigerant. The liquid refrigerant condensed by the condenser 118b may be transferred to the expander (not shown). The expander (not shown) may expand a high-temperature and high-pressure liquid refrigerant condensed in the condenser 118b to a low-pressure liquid refrigerant. For example, the expander (not shown) may include an expansion valve adapted to regulate a pressure difference of the refrigerant.

In an embodiment, the compressor 118c may compress the refrigerant in a gaseous state to be in a high-temperature and high-pressure state and may discharge the gaseous refrigerant in the high-temperature and high-pressure state. The discharged refrigerant may be transferred to the condenser 118b.

In an embodiment, the steam spray device 113 may receive water from the water reservoir 40 to form steam. The steam spray device 113 may include the steam generation device 114 connected to the water reservoir 40 via the connection member 41 to receive water to generate steam; the steam hose 115 adapted to guide the steam generated by the steam generation device 114 to the steam nozzle 116; and the steam nozzle 116 connected to the steam hose 115 and provided to spray steam into the clothing accommodation space 51.

In an embodiment, the steam nozzle 116 may be connected to an end of the steam hose 115 to spray steam toward the steam spray hole 70. In an embodiment, the steam nozzle 116 and the steam spray hole 70 may be disposed to be inclined in an upward direction (e.g., +Z direction) of the inner case 50 and may spray high-temperature and/or high-pressure steam toward the clothes C mounted in the clothing accommodation space 51.

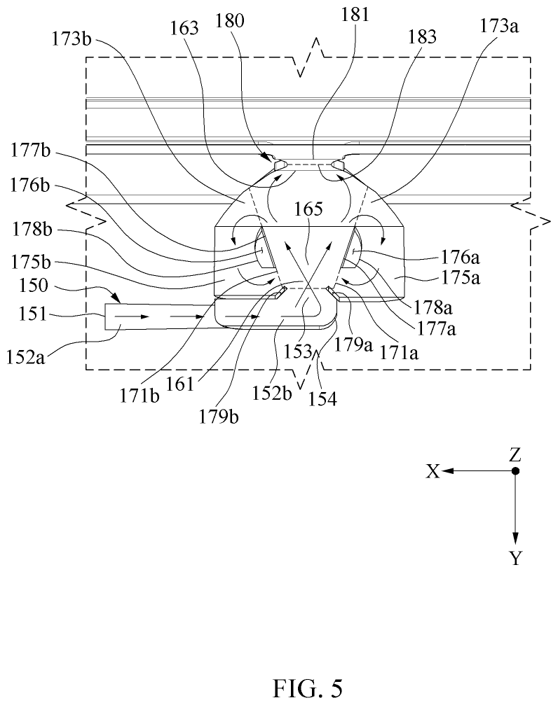

FIG. 4 is a perspective view of the steam nozzle 116 according to an embodiment of the present disclosure, and FIG. 5 is a top view of the steam nozzle 116 according to an embodiment of the present disclosure.

Referring to FIGS. 4 and 5, the steam nozzle 116 according to an embodiment may include at least some of a first fluid channel portion 150, a second fluid channel portion 180, a main fluid channel 165, and a side fluid channel 175.

In an embodiment, the steam nozzle 116 may be implemented with a combination of one or more configurations or features of the previously described embodiments unless it is technically obviously impossible.

In an embodiment, the first fluid channel portion 150, the second fluid channel portion 180, the main fluid channel 165, and the side fluid channel 175 may be virtual compartments that are virtually divided inside the steam nozzle 116 based on a factor relating to a movement direction of steam in the steam nozzle 116 (e.g., a direction in which steam moves or travels, a target receiving the steam, or a target to which the steam is transferred). However, actual implementations are not limited thereto, and the first fluid channel portion 150, the second fluid channel portion 180, the main fluid channel 165, and the side fluid channel 175 may be implemented as a single body but may overlap or be spaced apart in at least some areas.

Hereinafter, an internal structure of the steam nozzle 116 will be described with terms that are defined based on a primary movement direction of steam, but actual implementations are not limited thereto, and the movement direction of steam may include various movement directions in addition to the primary movement direction. Although the steam nozzle 116 of one embodiment of the present disclosure may have different shapes from ones in actual implementations, it may be construed that they are the same or homogeneous when they are terminologically identical or modified to the extent readily adaptable by a person having ordinary skill in the art.

In an embodiment, the first fluid channel portion 150 may be connected to the steam hose 115 to receive steam generated by the steam generation device 114. The first fluid channel portion 150 may include a first inlet 151 communicating with the steam hose 115 and a first outlet 153 communicating with the main fluid channel 165. The first fluid channel portion 150 may receive steam from the steam hose 115 and guide it into the main fluid channel 165.

In an embodiment, the first fluid channel portion 150 may include a movement region 152 that is a conduit through which steam moves, and the movement region 152 may guide a movement of the steam. In an embodiment, the movement region 152 may include a first area 152a and a second area 152b, which are a front end and a rear end relative to a travel direction of steam.

In an embodiment, the first area 152a may receive steam from the first inlet 151 and guide it to the second area 152b, and the second area 152b may receive steam from the first area 152a and guide it to the first outlet 153.

In an embodiment, the first area 152a may be a front end of the first fluid channel portion 150 to which the steam hose 115 is connected, and may extend in one direction (e.g., +/−X direction) to guide steam in a substantially straight direction. In an embodiment, the second area 152b may be a rear end of the first fluid channel portion 150 that connects the first area 152a of the first fluid channel portion 150 to the main fluid channel 165, and may have at least a portion that is bent to guide steam in a curved or angled line direction as the movement direction of the steam, thereby changing the movement direction of steam.

In an embodiment, the second area 152b may include a third side wall 154 that is bent to change the movement direction of steam traveling therein. The third side wall 154 may be disposed such that at least a portion of the third side wall 154 faces a direction in which the first area 152a extends (e.g., −X direction). The third side wall 154 may press at least a portion of the steam that has passed through the first area 152a in a different direction (e.g., −Y direction), and may change the movement direction of steam.

In an embodiment, the steam nozzle 116 may include a first open area 179a disposed between the third side wall 154 and the second fluid channel portion 180 and may be open to communicate with the outside of the steam nozzle 116. The first open area 179a may partition the second area 152b of the first fluid channel portion 150 from the main fluid channel 165, restrict a movement of steam, and change the movement direction of steam. In an embodiment, the steam nozzle 116 may have a second open area 179b provided in the first fluid channel portion 150 between a side wall opposite the third side wall 154 and the second fluid channel portion 180 and is open to communicate with the outside of the steam nozzle 116.

In an embodiment, the steam nozzle 116 may not include open areas (e.g., 179a and 179b), but may have damper members (not shown) at positions of the open areas 179a and 179b to change the movement direction of steam.

In an embodiment, the second area 152b may guide steam in a direction (e.g., +/−Y direction) different from that of the first area 152a. The second area 152b may change the movement direction of steam received from the first area 152a in one direction (e.g., −X direction) to a different direction, for example, a direction (e.g., −Y direction) of the clothing accommodation space 51.

In an embodiment, the movement direction of steam may be different in the first area 152a and the second area 152b, and the first fluid channel portion 150 may change in various ways the movement direction of steam received from the steam hose 115 through the first area 152a and the second area 152b. For example, the first area 152a may guide the steam in a first direction (e.g., −X direction), and the second area 152b may guide the steam in a second direction (e.g., −Y direction) that is different from the first direction.

In an embodiment, the second area 152b may have a cross-sectional area that is decreased in at least some areas as it extends in a direction (e.g., −Y direction) from the first area 152a toward the first outlet 153. When a cross-sectional area of a fluid channel decreases while a high-pressure fluid is flowing therethrough, a movement speed of the fluid in a corresponding area may decrease, and when the cross-sectional area increases after the fluid passes through it, the speed of the fluid may increase, allowing the fluid to be sprayed in a wide direction. For example, the movement speed of steam traveling in the main fluid channel 165 may decrease as the steam passes through the second area 152b, and increase again as the steam passes through the first outlet 153, allowing the steam to be sprayed in a wide direction.

In an embodiment, the second area 152b may have such a third side and such a shape with a decreasing cross-sectional area to change the movement direction of steam, and the steam that has passed through the first outlet 153 may spread and travel in a plurality of directions relative to a center direction (e.g., −Y direction).

In an embodiment, the second fluid channel portion 180 may receive steam from the main fluid channel 165 and spray the steam through the steam spray hole 70 toward the clothing accommodation space 51. The second fluid channel portion 180 may include a second inlet 183 communicating with the main fluid channel 165 and a second outlet 181 communicating with a spray area 71 of the steam spray hole 70.

In an embodiment, the main fluid channel 165 may be directly connected from the steam hose 115 to the steam spray hole 70, and the side fluid channel 175 may be branched from the main fluid channel 165 such that at least a portion of steam turns or is diverted inside the steam nozzle 116.

In an embodiment, the main fluid channel 165 may include a third area 165a disposed in a direction (e.g., X-Y plane direction) substantially parallel to a bottom surface (e.g., the bottom surface 50a in FIG. 3) of the inner case 50 to guide steam and a fourth area 165b disposed to be inclined at a predetermined angle from the bottom surface 50a of the inner case 50 to guide steam.

In an embodiment, steam in the main fluid channel 165 may move through the third area 165a in a direction (e.g., X-Y plane direction) substantially parallel to the bottom surface 50a, and may move through the fourth area 165b in a direction inclined at the predetermined angle from the bottom surface 50a.

In an embodiment, the second inlet 183 of the second fluid channel portion 180 may communicate with the fourth area 165b of the main fluid channel 165. The second fluid channel portion 180 may be disposed to be inclined at a predetermined angle substantially the same as or similar to the fourth area 165b, and the second outlet 181 may be disposed to be inclined at a predetermined angle from the bottom surface 50a to the inner case 50 to discharge steam slantingly in a vertical direction (e.g., +Z direction). For example, relative to a second direction (e.g., −Y direction) in which the second area 152b of the first fluid channel portion 150 guides steam, the fourth area 165b of the main fluid channel 165 and the second fluid channel portion 180 may discharge steam slantingly at a predetermined angle in a direction (e.g., +Z direction) perpendicular to the second direction.

In an embodiment, the steam spray hole 70 may be disposed to be slant toward a vertical direction (e.g., +/−Z direction) relative to the center of the inner case 50, for example, may be disposed adjacent to the bottom surface 50a of the inner case 50 close to the machine chamber. In an embodiment, when the second fluid channel portion 180 and the main fluid channel 165 are disposed to be inclined from the bottom surface 50a, the steam nozzle 116 may spray steam in a vertical direction (e.g., +Z direction) where the clothes C is located, and may thus improve the effectiveness of managing the clothes C.

In an embodiment, the second fluid channel portion 180 may have a cross-sectional area increasing in at least some areas as it extends in a direction from the second inlet 183 to the second outlet 181. In an embodiment, the main fluid channel 165 may have a cross-sectional area decreasing in at least some areas as it extends in a direction from the fourth area 165b to be adjacent to the second fluid channel portion 180. In this case, steam entering the second fluid channel portion 180 may have a decreased movement speed as it passes through the main fluid channel 165, and may have an increased movement speed again as it passes through the second fluid channel portion 180, and may thereby be sprayed in a wide direction.

In an embodiment, the second fluid channel portion 180 and the main fluid channel 165 may have decreased and increased cross-sectional areas to change the movement direction of steam, and steam that has passed through the second outlet 181 may be spread in a plurality of directions relative to the center direction, and the steam may be spread in a wider direction in the clothing accommodation space 51 to be evenly sprayed or distributed into the clothing accommodation space 51.

In an embodiment, the main fluid channel 165 may receive steam from the first fluid channel portion 150 and guide the steam to the second fluid channel portion 180. In an embodiment, the steam nozzle 116 may include the main fluid channel 165 and the side fluid channel 175 that are conduits for steam to travel inside the steam nozzle 116. The main fluid channel 165 may include a third inlet 161 and a third outlet 163 through which steam is transferred to or received from the outside of the main fluid channel 165.

In an embodiment, the main fluid channel 165 and the side fluid channel 175 may be virtual compartments that are virtually divided inside the steam nozzle 116 based on various factors, such as, a direction in which steam travels within the steam nozzle 116, a target receiving stream or target to which steam is to be transferred, and a function of changing a movement direction of steam. However, in actual implementations, the main fluid channel 165 and the side fluid channel 175 may be implemented as a single body or may overlap or be spaced apart in at least some areas.

In an embodiment, the main fluid channel 165 may be a center fluid channel through which steam moves from the first fluid channel portion 150 to the second fluid channel portion 180, and the main fluid channel 165 may communicate directly with the first fluid channel portion 150 and the second fluid channel portion 180, respectively. For example, the third inlet 161 may communicate with the first outlet 153 of the first fluid channel portion 150 to receive steam, the third outlet 163 may communicate with the second inlet 183 of the second fluid channel portion 180 to receive steam, and the main fluid channel 165 may be disposed between the third inlet 161 and the third outlet 163 to guide steam.

In an embodiment, the side fluid channel 175 may receive a portion of steam traveling from the main fluid channel 165 toward the second fluid channel portion 180, and may guide the steam toward the first fluid channel portion 150 to transfer the steam back to the main fluid channel 165.

In an embodiment, between the side fluid channel 175 and the main fluid channel 165, there may be a plurality of connection areas 171 and 173 that mutually transfer steam therebetween. In an embodiment, the first connection area 171 may be provided at a position adjacent to the third inlet 161 relative to the center of the main fluid channel 165, and the second connection area 173 may be provided in an area adjacent to the third outlet 163 relative to the center of the main fluid channel 165. For example, in the first connection area 171, steam that has passed through the side fluid channel 175 may move to the main fluid channel 165, and in the second connection area 173, at least a portion of steam that has passed through the main fluid channel 165 may move to the side fluid channel 175.

In an embodiment, the main fluid channel 165 may adjust a steam spray direction by allowing at least a portion of the steam moving from the first fluid channel portion 150 to the second fluid channel portion 180 to circulate in the main fluid channel 165 and the side fluid channel 175. The side fluid channel 175 may receive at least a portion of the steam passing through the main fluid channel 165, and guide it in a specific direction (e.g., +/−X direction) and transfer it back to the main fluid channel 165.

For example, as shown in FIG. 5, when the main fluid channel 165 receives steam in one direction (e.g., −Y direction) from the first fluid channel portion 150, and the main fluid channel 165 transfers the steam in one direction (e.g., −Y direction) to the second fluid channel portion 180, the main fluid channel 165 of the main fluid channel 165 may transfer the steam in a straight course extending primarily in the one direction (e.g., −Y direction). The side fluid channel 175 may receive at least a portion of steam from the main fluid channel 165, change the movement direction while moving it in a direction (e.g., +Y direction) opposite to the main fluid channel 165, change the movement direction of the steam to a new direction (e.g., +X and −Y), and transfer it back to the main fluid channel 165. The main fluid channel 165 may receive the steam traveling in a specific direction (e.g., +X direction) from the side fluid channel 175 and transfer it to the main fluid channel 165, allowing the steam nozzle 116 to spray steam widely in the specific direction (e.g., +X direction).

In another embodiment, when the steam nozzle 116 includes only the main fluid channel 165, excluding the side fluid channel 175, a majority of steam in the main fluid channel 165 may move in a straight course extending in one direction (e.g., −Y direction) from the first fluid channel portion 150 to the second fluid channel portion 180, and steam to be sprayed from the steam nozzle 116 may be sprayed in the straight course direction, which may restrict a steam spray range in other directions (e.g., +/−X direction).

In another embodiment, the main fluid channel 165 may include a pressure reducing member (not shown) therein to restrict a movement direction on the straight course, or to change the movement direction to an arrangement direction of the first fluid channel portion 150 and the second fluid channel portion 180 to change a steam transfer direction of the main fluid channel 165, the pressure of steam may be reduced as the steam passes through the main fluid channel 165, and a steam spray distance may be restricted.

In an embodiment, the steam nozzle 116 may include the main fluid channel 165 having a straight course from the first fluid channel portion 150 to the second fluid channel portion 180; and the side fluid channel 175 adapted to guide at least a portion of steam moving through the main fluid channel 165 to bypass and feed back into the main fluid channel 165, and steam sprayed through the steam nozzle 116 may thus be sprayed over a wide range with the spray pressure maintained or increased. As the steam is sprayed over a wide range, the clothing management device 100 including the steam nozzle 116 according to an embodiment may evenly spray steam onto the clothes C.

In an embodiment, the main fluid channel 165 may include a partitioning 176 that physically partitions the main fluid channel 165 and the side fluid channel 175. The partitioning 176 may be a wall surface provided inside the main fluid channel 165. The partitioning 176 may guide a movement direction of steam and may separate the main fluid channel 165 and the side fluid channel 175 from each other.

In an embodiment, the partitioning 176 may include a first side wall (e.g., 177a and 177b) facing the main fluid channel 165 and a second side wall (e.g., 178a and 178b) facing the side fluid channel 175, and may include a center area (e.g., 176a and 176b) disposed between the first side wall (e.g., 177a and 177b) and the second side wall (e.g., 178a and 178b). In an embodiment, the center area (e.g., 176a and 176b) between the first side wall (e.g., 177a and 177b) and the second side wall (e.g., 178a and 178b) may be open to communicate with the outside of the steam nozzle 116.

In an embodiment, the first side wall (e.g., 177a and 177b) may extend in a direction (e.g., −Y direction) from the first fluid channel portion 150 to the second fluid channel portion 180 and may be disposed to be inclined at a predetermined angle in a lateral direction (e.g., +/−X direction). The first side wall (e.g., 177a and 177b) may guide steam passing through the main fluid channel 165 to spread widely in the lateral direction and may guide at least a portion of the steam to travel to the side fluid channel 175 provided in the lateral direction.

In an embodiment, the second side wall (e.g., 178a and 178b) may extend in a direction (e.g., +Y direction) from the second fluid channel portion 180 to the first fluid channel portion 150 and may be disposed such that at least some areas are bent. The second side wall (e.g., 178a and 178b) may extend in a curved shape adjacent to the third outlet 163 to guide steam that has been guided into the side fluid channel 175 back toward the third inlet 161.

In an embodiment, the main fluid channel 165 may have a cross-sectional area increasing in at least some areas as it extends in a direction from the first fluid channel portion 150 to the second fluid channel portion 180. For example, as the first side wall (e.g., 177a and 177b) is disposed to be inclined in a lateral direction relative to the center of the main fluid channel 165, for example, as it is disposed to be inclined in a direction receding from the center of the main fluid channel 165, the cross-sectional area of the main fluid channel 165 may gradually increase. In an embodiment, as the cross-sectional area increases, the steam passing through the main fluid channel 165 may have a gradually increasing internal pressure, and the steam passing through the main fluid channel 165 may move at an increased movement speed to be distributed and moved into a wider range.

In an embodiment, the side fluid channel 175 may be bent in at least some areas as it extends in a direction (e.g., +Y direction) from the second fluid channel portion 180 to the first fluid channel portion 150 to detour steam in a direction (e.g., +/−X direction) different from the direction (e.g., +/−Y direction) in which the main fluid channel 165 extends. The side fluid channel 175 may guide the steam to travel inward and detour the steam in a direction (e.g., +/−X direction) receding from the main fluid channel 165, thereby varying the movement direction of steam. In this case, steam rejoining the main fluid channel 165 after passing through the side fluid channel 175 may travel in a different direction (e.g., +/−X direction) in addition to a travel direction (e.g., −Y direction) of the main fluid channel 165, and may thus have a wider movement range.

In an embodiment, the steam nozzle 116 may include a plurality of side fluid channels 175, and the plurality of side fluid channels 175 (e.g., 175a and 175b) may include a first side fluid channel 175a located on one side of the main fluid channel 165 and a second side fluid channel 175b located on another side opposite the one side of the main fluid channel 165.

In an embodiment, the plurality of side fluid channels 175a and 175b may be disposed on both lateral sides of the main fluid channel 165 to guide steam passing through the main fluid channel 165 to be widely sprayed in both lateral directions (e.g., +/−X direction). In an embodiment, the partitioning 176 may include first walls 177a and 178a partitioning the main fluid channel 165 and the first side fluid channel 175a and second walls 177b and 178b partitioning the main fluid channel 165 and the second side fluid channel 175b.

In an embodiment, the first side fluid channel 175a may include a plurality of connection areas 171a and 173a that mutually transfers steam together with the main fluid channel 165, and the first side fluid channel 175a and the main fluid channel 165 may be partitioned by the first walls 177a and 178a. The first walls 177a and 178a may include a first side wall 177a facing the main fluid channel 165 and a second side wall 178a facing the first side fluid channel 175a. A center area 176a between the first side wall 177a and the second side wall 178a may be open to communicate with the outside of the steam nozzle 116.

In an embodiment, the second side fluid channel 175b may include a plurality of connection areas 171b and 173b that mutually transfers steam together with the main fluid channel 165, and the first side fluid channel 175a and the main fluid channel 165 may be partitioned by the second walls 177b and 178b. The second walls 177b and 178b may include a first side wall 177b facing the main fluid channel 165 and a second side wall 178b facing the second side fluid channel 175b. A center area 176b between the first side wall 177b and the second side wall 178b may be open to communicate with the outside of the steam nozzle 116.

In an embodiment, in the steam nozzle 116, the main fluid channel 165 and the plurality of side fluid channels 175a and 175b may arise a vortex or swirl of at least a portion of steam passing through the inside of the main fluid channel 165. For example, the steam nozzle 116 may allow a portion of the steam that has passed through the first side fluid channel 175a to pass through the main fluid channel 165 to be transferred to the second side fluid channel 175b, and allow a portion of the steam that has passed through the second side fluid channel 175b to pass through the main fluid channel 165 to be transferred to the first side fluid channel 175a.

According to an embodiment, the steam nozzle 116 may include at least some of the various structures described above to increase a steam spray range, and may include various structures for increasing a steam spray pressure.

For example, the first fluid channel portion 150 may guide steam from the second area 152b to the first outlet 153. In an embodiment, the second area 152b may have a cross-sectional area decreasing in at least some areas as it extends toward the first outlet 153, and steam that has passed through the first outlet 153 and transferred to the main fluid channel 165 may have an increased spray pressure and a wider spray range. In an embodiment, the first fluid channel portion 150 may include the third side wall 154 to change a movement direction of steam. Through the third side wall 154, the steam may be guided to travel in a straight direction to the first outlet 153 of the first fluid channel portion 150, the main fluid channel 165 of the main fluid channel 165, and the second inlet 183 of the second fluid channel portion 180, which may increase the movement speed and the spray pressure of the steam.

For example, the second fluid channel portion 180 and the fourth area 165b of the main fluid channel 165 may be disposed to be inclined at a predetermined angle from the bottom surface 50a of the inner case 50 to guide steam such that the steam is sprayed in a vertical direction (e.g., +Z direction) of the inner case 50, thereby changing a movement direction of the steam and spraying the steam even to an area on top of the inner case 50.

For example, the steam nozzle 116 may include the main fluid channel 165 and the side fluid channel 175, and may include the partitioning 176 that includes a first side wall (e.g., 177a and 177b) facing the main fluid channel 165 and a second side wall (e.g., 178a and 178b) facing the side fluid channel 175 to physically partition the main fluid channel 165 and the side fluid channel 175. In an embodiment, the partitioning 176 may partition the main fluid channel 165 and the side fluid channel 175 and may form a course along which steam moves inside the main fluid channel 165, and may change a movement direction of steam traveling in the main fluid channel 165 and the side fluid channel 175 or change a pressure of the steam as the steam travels.

For example, the steam nozzle 116 may include a plurality of side fluid channels 175 (e.g., 175a and 175b), which may allow a portion of steam that has passed through one of the plurality of side fluid channels 175a and 175b to pass through the main fluid channel 165 to be transferred to another one of the plurality of side fluid channels 175a and 175b, thereby guiding at least a portion of the steam to swirl while the steam is moving inward.

For example, the main fluid channel 165 may primarily transfer steam in a straight course from the first fluid channel portion 150 to the second fluid channel portion 180 and may thereby increase a spray pressure of the steam, compared to a curved or bent course. In an embodiment, the side fluid channel 175 may receive a portion of steam of the main fluid channel 165, and change a movement direction of the steam such that the steam is spread in a lateral direction (e.g., +/−X direction) in addition to a direction (e.g., −Y direction) of the straight course to transfer the steam back to the main fluid channel 165. Accordingly, the main fluid channel 165 may increase the spray pressure of steam and increase the spray range of steam.

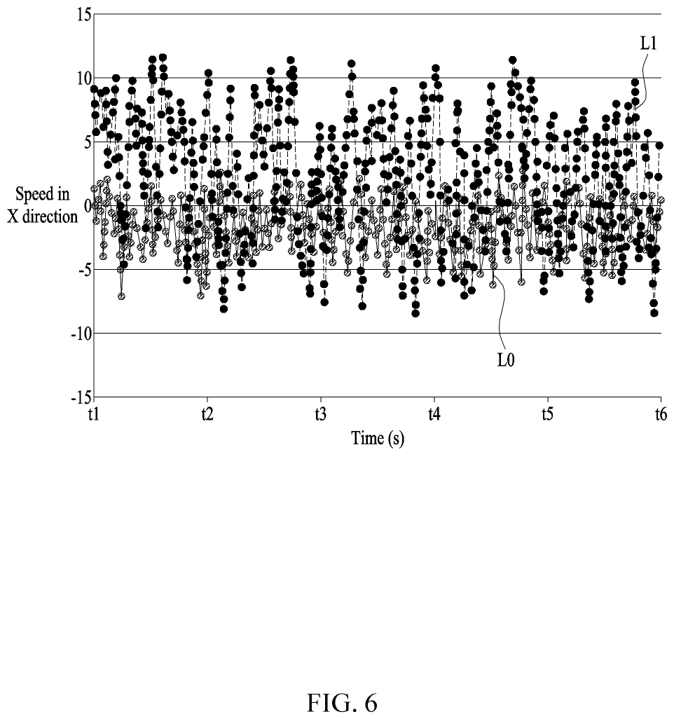

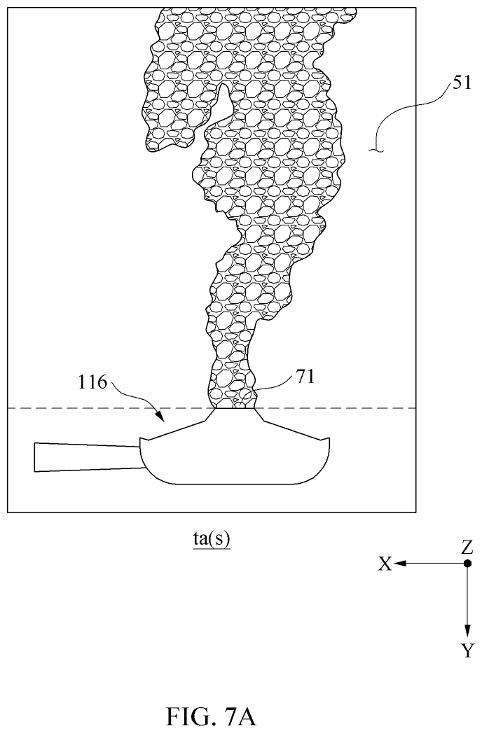

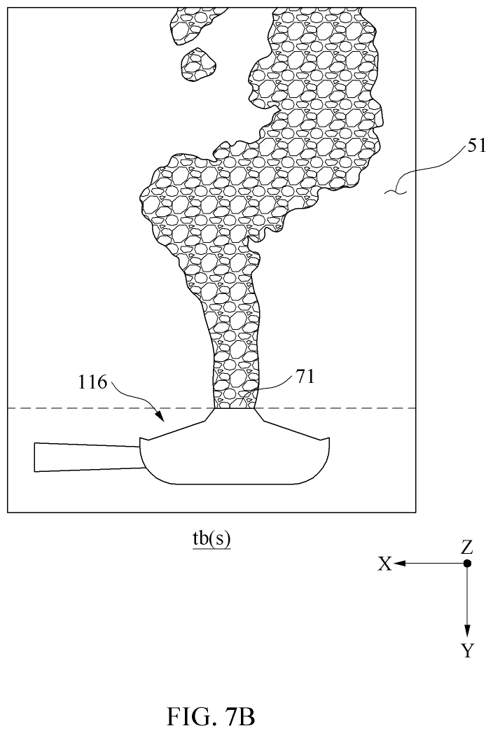

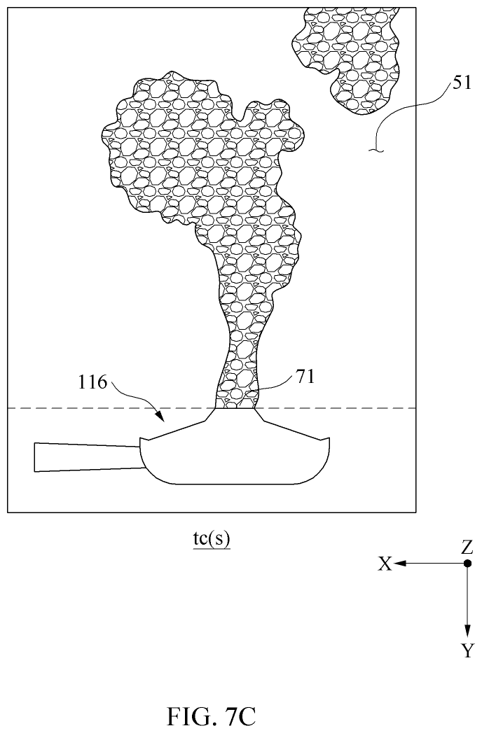

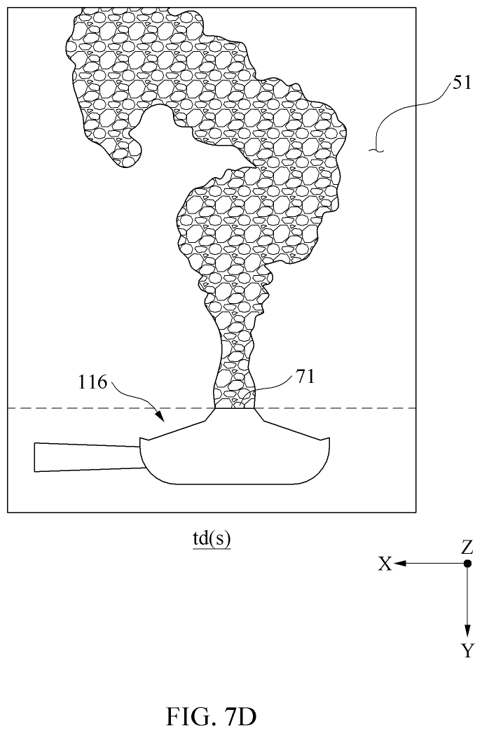

FIG. 6 is a graph illustrating a change in an X-direction spray speed of the steam nozzle 116 according to an embodiment of the present disclosure, and FIGS. 7A to 7D are diagrams illustrating various states in which the steam nozzle 116 sprays steam according to an embodiment of the present disclosure.

In an embodiment, the steam nozzle 116 may be implemented with a combination of one or more configurations or features of the previously described embodiments unless it is technically obviously impossible.

FIG. 6 is a graph profiling a speed at which steam of the steam spray device 113 is sprayed in an X direction (e.g., +/−X direction in FIG. 5) over time. In the graph of FIG. 6, L0 is a graph of the steam spray device 113 including a typical spray nozzle different from one shown in FIG. 5, and L1 is a graph of the steam spray device 113 including a spray nozzle shown in FIG. 5.

In FIG. 6, on a time axis, t1 to t6 may each be a time unit of 1 second or less. For example, t1 may be when 2 seconds have elapsed, t2 may be when 2.2 seconds have elapsed, t3 may be when 2.4 seconds have elapsed, t4 may be when 2.6 seconds have elapsed, t5 may be when 2.8 seconds have elapsed, and t6 may be when 3 seconds have elapsed, from a time point at which the steam spray device 113 starts spraying steam. On an x-direction speed axis, a unit of speed (or velocity) may be meters per second (m/s), and results of measuring a steam spray speed relative to the spray area 71 of the steam spray hole 70 of the steam spray device 113 or the third outlet 163 of the steam nozzle 116 may be shown. When the spray speed from the spray area 71 increases, the spray pressure may increase, and thus the steam may be sprayed relatively farther in a spray direction.

Referring to FIG. 6, L1, the graph for the steam spray device 113 of an embodiment of the present disclosure, may show that the steam spray speed in the X direction over time may change greatly compared to L0. For example, L0 shows that a speed range is limited to a range of +/−5 in the X direction, while L1 shows that steam may be sprayed in the X direction at a speed of up to +/−10.

In an embodiment, the steam spray device 113 may spray steam in a wider range from a straight direction where the steam is mainly sprayed to both lateral directions, and the clothing management device 100 may thus spray steam on various parts of the clothes C located in a wide range.

FIGS. 7A to 7D are diagrams illustrating states in which the steam spray device 113 of the L1 graph of FIG. 6 sprays steam. ta to td may each be a time unit of 0.1 seconds or less and may show, for example, sequential states in which the steam nozzle 116 sprays steam during a time between t5 and t6 of FIG. 6. Here, ta may be when 2.87 seconds have elapsed, tb may be when 2.88 seconds have elapsed, te may be when 2.89 seconds have elapsed, and td may be when 2.90 seconds have elapsed, from a time point at which the steam spray device 113 starts spraying steam.

Referring to FIGS. 7A to 7D, it may be shown that steam is sprayed slantingly and sequentially in both lateral directions (e.g., +/−X direction) relative to a center direction (e.g., −Y direction) for a time from ta to td. In an embodiment, the steam spray device 113 may spray steam over a wide range by changing a steam spray speed in an X direction in a relatively short time unit.

Hereinafter, a steam spray process of the steam spray device 113 of FIGS. 6 through 7D will be described in detail with reference to a travel direction of steam shown in FIG. 5.

In an embodiment, when a steam travel direction from the first fluid channel portion 150 to the second fluid channel portion 180 is set to a main direction (e.g., −Y direction), steam to be transferred from the first fluid channel portion 150 to the main fluid channel 165 may be guided to be inclined in one direction (e.g., +X direction) of lateral directions (e.g., +/−X direction) perpendicular to the main direction by the third side wall 154.

In an embodiment, at least a portion of steam passing from the first fluid channel portion 150 into the main fluid channel 165 may be transferred to the second fluid channel portion 180, and another portion of the steam may be transferred to a plurality of side fluid channels 175a and 175b. In this case, at least a portion of the steam may be transferred to a second side fluid channel 175b located in an inclined direction (e.g., +X direction) by the third side wall 154. The second side fluid channel 175b may change the steam travel direction to be inclined in the other lateral direction (e.g., −X direction) and transfer the steam back to the main fluid channel 165.

In an embodiment, a portion of steam that has passed through the second side fluid channel 175b and the main fluid channel 165 may be transferred to the second fluid channel portion 180, and another portion of the steam may be transferred to the plurality of side fluid channels 175a and 175b. In this case, at least a portion of the steam may be transferred to the first side fluid channel 175a located in an inclined direction (e.g., −X direction) by the second side fluid channel 175b. The first side fluid channel 175a may change the steam travel direction to be inclined in the other lateral direction (e.g., +X direction) and transfer the steam back to the main fluid channel 165.

In an embodiment, the steam nozzle 116 may allow steam to move alternately in one direction and another direction of both lateral directions (e.g., +/−X direction) with greater pressure while moving inside the main fluid channel 165, and the steam nozzle 116 may change a steam spray direction according to time to allow the steam to be sprayed in a wider range while vibrating in both lateral directions.

According to an embodiment, a clothing management device 100 may include: an outer case 10 forming an exterior; an inner case 50 disposed inside the outer case 10 and providing a clothing accommodation space 51; a steam generation device 114 adapted to generate steam by heating water; a steam nozzle 116 coupled to the inner case 50 to spray steam; and a steam hose 115 connecting the steam generation device 114 and the steam nozzle 116, wherein the steam nozzle 116 may include: a steam spray hole 70 adapted to spray steam into the clothing accommodation space 51; a main fluid channel 165 directly connected from the steam hose 115 to the steam spray hole 70; and a side fluid channel 175 branched from the main fluid channel 165 to allow at least a portion of steam to turn inside the steam nozzle 116.

In an embodiment, the steam nozzle 116 may include a partitioning 176 physically partitioning between the main fluid channel 165 and the side fluid channel 175, and the partitioning 176 may include a first side wall (e.g., 177a and 177b) facing the main fluid channel 165 and a second side wall (e.g., 178a and 178b) facing the side fluid channel 175.

In an embodiment, the partitioning 176 may have a center area (e.g., 176a and 176b) between the first side wall (e.g., 177a and 177b) and the second side wall (e.g., 178a and 178b) that is open to communicate with the outside of the steam nozzle 116.

In an embodiment, the first side wall (e.g., 177a and 177b) may be disposed to be inclined at a predetermined angle in a direction receding from a center of the main fluid channel 165.

In an embodiment, the second side wall (e.g., 178a and 178b) may have at least a portion bent to allow steam inside the side fluid channel 175 to turn.

In an embodiment, the main fluid channel 165 may a cross-sectional area that increases as it is developed from the steam hose 115 to the steam spray hole 70.

In an embodiment, the steam nozzle 116 may include a plurality of side fluid channels 175, and the plurality of side fluid channels (e.g., 175a and 175b) may include a first side fluid channel 175a disposed on one side of the main fluid channel 165 and a second side fluid channel 175b disposed on another side opposite the one side of the main fluid channel 165.

In an embodiment, the steam nozzle 116 may be adapted to transfer a portion of steam that has passed through the first side fluid channel 175a to the second side fluid channel 175b through the main fluid channel 165 such that at least a portion of the steam swirls while moving therein.

In an embodiment, the steam nozzle 116 may include a first fluid channel portion 150 connected to the steam hose 115 to receive steam and guide the steam into the main fluid channel 165.

In an embodiment, the first fluid channel portion 150 may include: a first inlet 151 communicating with the steam hose 115; a first area 152a receiving steam from the first inlet 151 and guiding the steam; a second area 152b receiving steam from the first area 152a and guiding the steam in a direction different from that of the first area 152a; and a first outlet 153 receiving steam from the second area 152b and communicating with the main fluid channel 165.

In an embodiment, the second area 152b may include a third side wall 154 that is bent to change a movement direction of steam moving therein.

In an embodiment, the second area 152b may have cross-sectional area that decreases in at least a portion thereof as it extends in a direction from the first area 152a to the first outlet 153.

In an embodiment, the main fluid channel 165 may include a third area 165a disposed substantially parallel to a bottom surface 50a of the inner case 50 to guide steam, and a fourth area 165b disposed to be inclined at a predetermined angle from the bottom surface 50a of the inner case 50 to guide steam.

In an embodiment, the steam nozzle 116 may include a second fluid channel portion 180 receiving steam from the main fluid channel 165 and guiding the steam to the steam spray hole 70, and the second fluid channel portion 180 may include a second inlet 183 communicating with the fourth area 165b of the main fluid channel 165 and a second outlet 181 adapted to discharge steam slantingly at a predetermined angle from the bottom surface 50a of the inner case 50.

According to an embodiment, a steam spray device 113 may include: a steam generation device 114 adapted to generate steam by heating water; a steam hose 115 connected to the steam generation device 114; and a steam nozzle 116 connected to the steam hose 115 to receive steam and spray the steam to the outside, wherein the steam nozzle 116 may include: a steam spray hole 70 adapted to spray steam into a clothing accommodation space 51; a main fluid channel 165 directly connected from the steam hose 115 to the steam spray hole 70; a side fluid channel 175 branched from the main fluid channel 165 to allow at least a portion of steam to turn inside the steam nozzle 116; and a partitioning 176 including a first side wall (e.g., 177a and 177b) facing the main fluid channel 165 and a second side wall (e.g., 178a and 178b) facing the side fluid channel 175, and partitioning between the main fluid channel 165 and the side fluid channel 175.

In an embodiment, the first side wall (e.g., 177a and 177b) may be disposed to be inclined at a predetermined angle in a direction receding from a center of the main fluid channel 165, and the second side wall (e.g., 178a and 178b) may be at least partially bent to allow steam inside the side fluid channel 175 to turn.

In an embodiment, the steam nozzle 116 may include a plurality of side fluid channels 175 each adapted to transfer a portion of steam that has passed through one of the plurality of side fluid channels (e.g., 175a and 175b) to another fluid channel of the plurality of side fluid channels (e.g., 175a and 175b) through the main fluid channel 165 such that at least a portion of the steam swirls while moving therein.

In an embodiment, the steam nozzle 116 may include a first fluid channel portion 150 connected to the steam hose 115 to receive steam and guide the steam into the main fluid channel 165; and a second fluid channel portion 180 receiving steam from the main fluid channel 165 and guiding the steam into the steam spray hole 70.

In an embodiment, the first fluid channel portion 150 may include a first inlet 151 communicating with the steam hose 115; a first area 152a receiving steam from the first inlet 151 and guiding the steam in a first direction; a second area 152b receiving steam from the first area 152a and guiding the steam in a second direction different from the first direction; and a first outlet 153 receiving steam from the second area 152b and communicating with the main fluid channel 165.

In an embodiment, the second fluid channel portion 180 may include a second inlet 183 communicating with the main fluid channel 165 and a second outlet 181 adapted to discharge steam slantingly at a predetermined angle relative to a direction perpendicular to the second direction.

While the disclosure has been shown and described with reference to various embodiments thereof, it will be understood that the various embodiments are intended to be illustrative, not limiting. It will further be understood by those skilled in the art that various changes in form and details may be made without departing from the true spirit and full scope of the disclosure as defined by the appended claims and their equivalents.

What is claimed is:

1. A clothing management device, comprising:
an outer case forming an exterior of the clothing management device;
an inner case arrangeable inside the outer case and in which a clothing accommodation space is provided;
a steam generation device to generate steam by heating water;
a steam nozzle to be coupled to the inner case to spray steam; and
a steam hose to connect the steam generation device and the steam nozzle,
wherein the steam nozzle comprises:
a steam spray hole to spray steam generated by the steam generation device into the clothing accommodation space;
a main fluid channel directly connectable between the steam hose and the steam spray hole; and
a side fluid channel branched from the main fluid channel to allow at least a portion of the steam to turn a direction in which the steam is moved inside the steam nozzle.

2. The clothing management device of claim 1, wherein the steam nozzle comprises:
a partitioning between the main fluid channel and the side fluid channel,
wherein the partitioning comprises:
a first side wall facing the main fluid channel; and
a second side wall facing the side fluid channel.

3. The clothing management device of claim 2, wherein a center area between the first side wall and the second side wall is open to allow the steam to be sprayed to an outside of the steam nozzle.

4. The clothing management device of claim 2, wherein the first side wall is inclined at a predetermined angle in a direction receding from a center of the main fluid channel.

5. The clothing management device of claim 2, wherein the second side wall has at least a portion bent to allow steam inside the side fluid channel to turn a direction in which the steam moves.

6. The clothing management device of claim 1, wherein the main fluid channel has a cross-sectional area that increases from the steam hose to the steam spray hole.

7. The clothing management device of claim 1, wherein the steam nozzle comprises a plurality of side fluid channels,
wherein the plurality of side fluid channels comprise:
a first side fluid channel on one side of the main fluid channel; and
a second side fluid channel on another side of the main fluid channel opposite the one side of the main fluid channel.

8. The clothing management device of claim 7, wherein the steam nozzle transfers a portion of steam that has passed through the first side fluid channel to the second side fluid channel through the main fluid channel such that at least a portion of the steam swirls while the steam moving therein.

9. The clothing management device of claim 1, wherein the steam nozzle comprises a fluid channel portion to be connected to the steam hose to receive steam and guide the steam into the main fluid channel.

10. The clothing management device of claim 9, wherein the fluid channel portion comprises:
a first inlet connectable with the steam hose;
a first area to receive steam from the first inlet and guide the steam;
a second area to receive the steam from the first area and guide the steam in a direction different from that of the first area; and
a first outlet to receive the steam from the second area and allow the steam to move to the main fluid channel.

11. The clothing management device of claim 10, wherein the second area comprises:
a third side wall bent to change a movement direction of the steam moving in the second area.

12. The clothing management device of claim 10, wherein the second area has a cross-sectional area that decreases in at least a portion that extends in a direction from the first area to the first outlet.

13. The clothing management device of claim 1, wherein the main fluid channel comprises:
a third area arrangeable substantially parallel to a bottom surface of the inner case to guide steam, and a fourth area arrangeable to be inclined at a predetermined angle from the bottom surface of the inner case to guide steam.

14. The clothing management device of claim 13, wherein the steam nozzle comprises:
a first fluid channel portion connected to the steam hose to receive steam and guide the steam into the main fluid channel, and
a second fluid channel portion to receive steam from the main fluid channel and guide the steam into the steam spray hole,
wherein the second fluid channel portion comprises:
a second inlet to the fourth area of the main fluid channel; and
a second outlet to discharge steam slantingly at a predetermined angle from the bottom surface.

15. A steam spray device, comprising:
a steam generation device to generate steam by heating water;
a steam hose to connect to the steam generation device; and
a steam nozzle to be connected to the steam hose to receive steam generated by the steam generation device and spray the steam an outside of the steam nozzle,
wherein the steam nozzle comprises:
a steam spray hole to spray steam to the outside of the steam nozzle;
a main fluid channel directly connectable between the steam hose and the steam spray hole;
a side fluid channel branched from the main fluid channel to allow at least a portion of the steam to turn a direction in which the steam moves inside the steam nozzle; and
a partitioning comprising a first side wall facing the main fluid channel and a second side wall facing the side fluid channel, and the partitioning being between the main fluid channel and the side fluid channel.

16. The steam spray device of claim 15, wherein the first side wall is inclined at a predetermined angle in a direction receding from a center of the main fluid channel, and
the second side wall is at least partially bent to allow steam inside the side fluid channel to turn a direction in which the steam moves.

17. The steam spray device of claim 15, wherein the steam nozzle comprises:
a plurality of side fluid channels to transfer a portion of steam that has passed through one of the plurality of side fluid channels to another fluid channel of the plurality of side fluid channels through the main fluid channel such that at least a portion of the steam swirls while moving therein.

18. The steam spray device of claim 15, wherein the steam nozzle comprises:
a first fluid channel portion connected to the steam hose to receive steam and guide the steam into the main fluid channel; and
a second fluid channel portion receiving steam from the main fluid channel and guiding the steam into the steam spray hole.

19. The steam spray device of claim 18, wherein the first fluid channel portion comprises:
a first inlet connectable with the steam hose;
a first area to receive steam from the first inlet and guide the steam along a first direction;
a second area to receive the steam from the first area and guide the steam along a second direction different from the first direction; and
a first outlet to receive the steam from the second area and allow the steam to move to the main fluid channel.

20. The steam spray device of claim 19, wherein the second fluid channel portion comprises:
a second inlet to allow steam to move from the main fluid channel; and
a second outlet to discharge steam slantingly at a predetermined angle relative to a direction perpendicular to the second direction.
